SEQUENCE LISTING (8 sequences)

SEQ ID NO: 1 - DNA, 23 bp, Mus musculus
gaattcccatggctggacacctg

SEQ ID NO: 2 - DNA, 23 bp, Mus musculus
gcggccgctcagtttgaatgcat

SEQ ID NO: 3 - DNA, 23 bp, Mus musculus
gaattcgcatgtataacatgatg

SEQ ID NO: 4 - DNA, 24 bp, Mus musculus
gcggccgctcacatgtgcgacagg

SEQ ID NO: 5 - DNA, 23 bp, Mus musculus
gaattcggctggatttcctttgg

SEQ ID NO: 6 - DNA, 23 bp, Mus musculus
gcggccgcttatgcaccagagtt

SEQ ID NO: 7 - DNA, 23 bp, Mus musculus
gaattcacatggctgtcagcgac

SEQ ID NO: 8 - DNA, 24 bp, Mus musculus
gcggccgcttaaaagtgcctcttc

(19) United States
(12) Patent Application Publication
(10) Pub. No.: US 2011/0129928 A1
(43) Pub. Date: Jun. 2, 2011

(54) Method of Manufacturing Induced Pluripotent Stem Cell Originated from Somatic Cell

(75) Inventors: Se Pill Park, Jeju-do (KR); Eun Young Kim, Seoul (KR); Kilsoo Jeon, Gyeonggi-do (KR); Ssang-Goo Cho, Seoul (KR)

(73) Assignee: Mirae Biotech Co., Ltd., Gwangjin-gu (KR)

(21) Appl. No.: 12/865,690

(22) PCT Filed: Jan. 31, 2008
(86) PCT No.: PCT/KR08/00585

§ 371 Date: Nov. 22, 2010

(30) Foreign Application Priority Data
Jan. 30, 2008 (KR) 10-2008-0009755

(51) Int. Cl. C12N 15/86

(52) U.S. Cl. 435/456

(57) ABSTRACT

Disclosed is a method for manufacturing stem cells including preparing Oct-4 gene, Sox2 gene, C-myc gene, and Klf-4 gene from mouse embryonic stem cells, and allowing each of the genes to be infected in host cells using a lentiviral vector system to generate viruses in which each of the genes are induced; concentrating or mixing each of the viruses to prepare a virus concentrated mixture, and mixing the virus concentrated mixture and a first culture solution to prepare a virus solution; floating mouse somatic cells having been cultivated in advance in a first culture dish, and mixing and reacting the floated somatic cells and the virus solution to prepare a somatic cell-virus mixture; adding and retaining the somatic cell-virus mixture as is in a second culture dish including a second culture solution to induce the genes in the somatic cells; and cultivating the somatic cells.

### TECHNICAL FIELD

The present invention relates to a method of manufacturing induced pluripotent stem cells originated from somatic cells, and more particularly, to a method of manufacturing of induced pluripotent stem cells originated from somatic cells which may dramatically effectively manufacture the induced pluripotent stem cells originated form somatic cells.

### BACKGROUND ART

Embryonic Stem (ES) cells originated from inner cell masses of blastocyst of mammalia may branch out about two hundred ten organs of the human and have characteristics of being endlessly proliferated while maintaining pluripotency.

Accordingly, human ES cells may be expected to be used for disease studies, efficiency/stability test of drugs, diseases treatment (childhood diabetes, spinal damage), and the like.

However, the use of human embryos for the purpose of manufacturing the ES cells raises ethical debates, and disadvantageously has limitations due to a significantly less probability of manufacturing stem cells for specific patients and specific diseases.

### DISCLOSURE OF INVENTION

### Technical Goals

An aspect of the present invention provides a method of manufacturing of induced pluripotent stem cells originated from somatic cells which may dramatically effectively manufacture the induced pluripotent stem cells originated form somatic cells.

### Technical Solutions

According to an aspect of the present invention, there is provided a method for manufacturing stem cells, the method including: preparing Oct-4 gene, Sox2 gene, C-myc gene, and Klf-4 gene from mouse embryonic stem cells, and allowing each of the genes to be infected in host cells using a lentiviral vector system to generate viruses in which each of the genes are induced; concentrating or mixing each of the viruses to prepare a virus concentrated mixture, and mixing the virus concentrated mixture and a first culture solution to prepare a virus solution; floating mouse somatic cells having been cultivated in advance in a first culture dish, and mixing and reacting the floated somatic cells and the virus solution to prepare a somatic cell-virus mixture; adding and retaining the somatic cell-virus mixture as is in a second culture dish including a second culture solution to induce the genes in the somatic cells; and cultivating the somatic cells in which the genes are induced in a third culture dish including a third culture solution.

In this instance, the allowing of each of the genes to be infected in host cells may include preparing the Oct-4 gene, the Sox2 gene, the C-myc gene, and the Klf-4 gene from the mouse embryonic stem cells to clone the genes in a lentiviral vector, respectively; and allowing the cloned lentiviral vectors to be infected in the host cells to generate viruses in which the genes are induced by the cloned lentiviral vectors, respectively.

Also, the concentrating of each of the viruses may be achieved by centrifugation, and the mixing of each of the viruses may be performed in such a manner that an amount of each of the viruses is the same.

Also, the virus concentrated mixture and the first culture solution may be mixed with a ratio of about 1:1 to 5.

Also, the floating of mouse somatic cells may include separating the somatic cells from the first culture dish using a cell separation solution; and centrifuging the separated somatic cells.

Also, a volume ratio of the somatic cell-virus mixture and the second culture solution may be about 1:10 to 20.

Also, composition of the first and second culture solutions may be the same.

Also, the reacting may be performed for about 5 to 15 minutes.

### BRIEF DESCRIPTION OF DRAWINGS

FIG. 1 is a photograph showing a DNA band detected by electrophoresis after performing a Polymerase Chain Reaction (PCR);

FIG. 2 is a schematic diagram illustrating a mechanism of pGEM-T easy vector;

FIG. 3 is an electrophoretic photograph showing genes cloned in T-vector;

FIG. 4 is a schematic diagram illustrating a mechanism of pENTR4 vector;

FIG. 5 is a schematic diagram illustrating homologous recombination according to an exemplary embodiment of the present invention;

FIG. 6 is a mimetic diagram illustrating an envelope plasmid, a packaging plasmid, and a target vector each for producing viruses;

FIG. 7 is a microphotograph illustrating a state where a lentiviral vector is infected in a 239T cell;

FIG. 8 is a microphotograph (A) and a fluorescence microphotograph (B) each showing stem cells 24 hours after inducing genes;

FIG. 9 is a fluorescence microphotograph showing stem cells 48 hours after inducing genes by Comparative Example (A) and Example (B);

FIG. 10 is a microphotograph (A) and a fluorescence microphotograph (B) each showing stem cells 10 days after inducing genes;

FIG. 11 is an electrophoretic photograph showing gene expression within stem cells originated from somatic cells;

FIG. 12 is a microphotograph showing stem cells in which an Alkaline phosphatase (AP) is activated;

FIG. 13 is microphotographs showing a state of SSEA-1 expression;

FIG. 14 is microphotographs showing a state of Oct-4 expression;

FIG. 15 is a microphotograph (A) and a fluorescence microphotograph (B) each showing the differentiation of stem cells where differentiation has been induced for three days; and

FIG. 16 is fluorescence microphotographs showing the differentiation of stem cells where differentiation has been induced for seven days.

### BEST MODE FOR CARRYING OUT THE INVENTION

Reference will now be made in detail to embodiments of the present invention, examples of which are illustrated in the accompanying drawings, wherein like reference numerals refer to the like elements throughout. The embodiments are described below in order to explain the present invention by referring to the figures.

The present invention relates to a method for establishing induced pluripotent stem cells originated from somatic cells by inducing genes specifically over-expressed in stem cells to somatic cells, unlike the somatic cells, to thereby cause de-differentiation of the somatic cells.

Specifically, the present invention may directly induce a reprogramming process in the somatic cells having been differentiated to thereby successfully manufacture stem cells having pluripotency.

Four transcription factors related to the above are Oct3/4, Sox2, c-Myc, and Klf4. The Oct3/4 and Sox2 are main transcription factors determining the pluripotency, which may function to up-regulate genes concerning stemness and suppress genes concerning the differentiation. The c-Myc and Klf4 may change a structure of chromatin to thereby enable the Oct3/4 and Sox2 to be successfully combined with target genes

According to the present invention, in order to justify a method for effectively inducing the above-mentioned four genes into somatic cells, a lentiviral vector may be used, and presence/absence of adhesiveness of cell may be regulated at the time of inducing the genes to thereby maximize manufacturing efficiency of stem cells.

According to the present invention, in order to manufacture stem cells, the four transcription factors, that is, Oct-4 gene, Sox2 gene, C-myc gene, and Klf-4 gene are required to be generated from mouse embryonic stem cells.

For this purpose, total RNA may be extracted from the mouse embryonic stem cells and cDNA may be composed from the extracted total RNA. The composed cDNA may be cloned by predetermined primers and amplified by RT-PCT.

The prepared transcription factors may be cloned in a T-vector, and then the cloned transcription factors may be sub-cloned in an entry cloning vector such as a pENTR4 vector (manufactured by Invitrogen) so that the cloned transcription factors are again homologus recombinated with the lentiviral vector.

Each of the transcription factors cloned in the T-vector may be ligated with the entry cloning vector to thereby be sub-cloned in the entry cloning vector.

The entry cloning vector in which the transcription factors are sub-cloned may be induced into the lentiviral vector through the homologus recombination with the lentiviral vector.

The lentiviral vectors of four types including the respective transcription factors may be infected by respective viruses to generate transgenic viruses in which respective genes are induced.

The viruses of the present invention as described above may be generated by a lentiviral vector system.

Each of the four type-viruses in which the four type-genes are effectively induced may be concentrated to prepare a virus concentrated mixture. The above-described concentration process may be performed by centrifuging the respective viruses. Through the concentration process, gene transfer efficiency into mouse somatic cells which will be described below may be significantly increased.

A content of each viruses for preparing the virus concentrated mixture may be preferably maintained to be identical to each other so that the four genes are effectively expressed.

The prepared virus concentrated mixture may be mixed with a first culture solution to thereby prepare a virus solution. A mixture ratio between the virus concentrated mixture and the first culture solution is about 1:1 to 5.

When the mixture ratio of the first culture solution to the virus concentrated mixture exceeds 5, the gene transfer efficiency may be significantly reduced. Conversely, when the mixture ratio thereof is less than 1, problems may occur in stability of somatic cells, that is, objects of the gene transfer.

The mixture ratio between the virus concentrated mixture and the first culture solution is preferably about 1:1.

The mouse somatic cells in which the four type-gene combinations will be induced may be cultivated in advance in a first culture dish before performing the gene transfer, and attached on the first culture dish.

For the gene transfer, a cell separation solution such as a triple solution and the like is required to be used in the first culture dish where the mouse somatic cells are cultivated to thereby separate the somatic cells from the first culture dish and float the separated somatic cells. The floated somatic cells may be separated and prepared only with a solid content of the somatic cells by performing centrifugation.

The purpose of floating the somatic cells is to increase a reaction surface area between the virus solution and the cells. A time required when the floated cells are completely attached on the culture dish may be about two and three hours. According to the present invention, superior efficiency may be acquired along with an increase in a probability that viruses are penetrated into spherical cells in three-dimensions, in comparison with a method of gene transfer of attached somatic cells.

The floated somatic cells and virus solution as described above may be reacted with each other for about 5 or 15 minutes after being added to a reaction dish such as a conical tube and the like and mixed together.

A somatic cell-virus mixture may be prepared through the reaction.

The somatic cell-virus mixture is moved to a second culture dish including a second culture solution and retained as is for about 24 hours, and thereby infection of the somatic cells may be achieved. Specifically, genes included in the viruses may be induced into the somatic cells.

A ratio of the second culture solution to the somatic cell-virus mixture may be preferably about 10 to 20:1. When the ratio thereof exceeds ‘20’, the gene transfer efficiency of the somatic cells may be deteriorated.

Also, a composition of the first culture solution and second culture solution may be preferably the same, and thereby gene expression may be facilitated by maintaining metabolism and function of the somatic cells

The somatic cells in which gene transfer is carried out in about 24 hours may be separated from the second culture dish using the cell separation solution, and moved to a third culture dish including a third culture solution to be cultivated for several weeks, and thereby obtaining stem cells.

A basic composition of the third culture solution is the same as the first culture solution and the second culture solution, however, additionally includes Foetal Bovine Serum (FBS) and undifferentiated inducer and the like.

Hereinafter, the present invention will be described in detail by examples. It is to be understood, however, that these examples are for illustrative purpose only, and are not construed to limit the scope of the present invention.

### Example

### 1. Preparation of T-Vector in which Oct4, Sox2, C-myc and Klf4 Genes are Induced

(1) Total RNA Extraction from Mouse Embryonic Stem Cells

1 ml of a trizol reagent (manufactured by Sigma) was inserted in recovered embryonic stem cells and retained as was for five minutes at room temperature to destruct the cells, thereby eluting contents of the cells. Next, 200 μl of chloroform was inserted, mixed together in an inverted state, retained as was for about 15 minutes at a room temperature, and then centrifuged under a condition of 1,300 rpm, 15 minutes, and 4° C., thereby collecting only a supernatant except for precipitation, that is, solid of DNA and protein. Next, 500 μl of isopropanol was inserted in the obtained mixture, retained at a room temperature for about 10 minutes, centrifuged under a condition of 1,300 rpm, 10 minutes, and 4° C., thereby removing the remaining chloroform. Next, the obtained mixture was washed using 1 ml of EtOH of 75%, centrifuged under a condition of 8,000 rpm, 5 minutes, and 4° C., removed a supernatant, and dried pellet. Next, the pellet was melted in 20 μl of an RNA inhibitor (diethylpyrocarbonate (DEPC) water), thereby preparing a total RNA.

(2) cDNA Composition

In order to compose cDNA, 3 μl of the total RNA and 2 ul of oligo dT(dT) were mixed together, reacted for about 5 minutes at 70° C., and retained as was for about 5 minutes at 4° C. 15 ul of a reverse transcription mixture (5.6 ul of water, 4 ul of ImProm-II 5× buffer, 2.4 ul of 25 mM MgCl₂, 1 ul of 10 mM dNPT, 1 ul of RNasin Ribonuclease inhibitor, and 1 ul of Improm-II reverse transcriptase, manufactured by Promega) was inserted in the obtained mixture, annealed for about 5 minutes at 25° C., extended for 60 minutes at 37° C., and inactivated the Improm-II reverse transcriptase for about 15 minutes at 70° C.

(3) RT-PCR

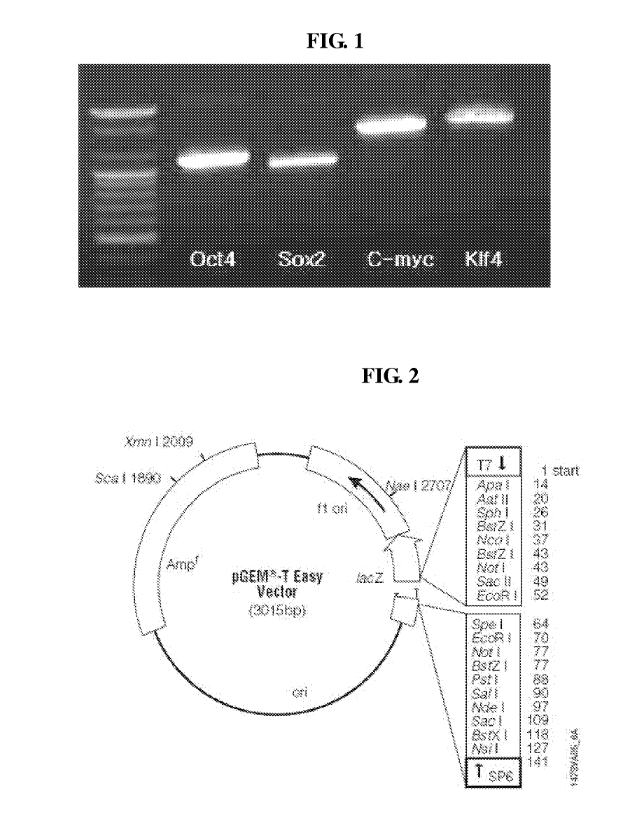

The obtained mixture was extended with 30 cycles for 15 minutes at 95° C., 1 minute at 95° C., 1 minute at 51 to 53° C., 1 minute at 72° C., and 5 minutes at 72° C. using the composed cDNA (product name: AccuPrime DNA Taq polymerase, manufactured by Invitrogen). A primer used for gene cloning was mOct4 (forward primer: 5′-GAATTC-CCATGGCTGGACACCTG-3′ (23mer), reverse primer: 5′-GCGGCCGC-TCAGTTTGAATGCAT-3′ (23mer)), mSox2(forward primer: 5′-GAATTC-GCATGTATAACATGATG-3′ (23mer)), reverse primer: 5′-GCGGCCGC-TCACATGTGCGACAGG-3′ (24mer), mC-myc(forward primer: 5′-GAATTC-GGCTGGATTTCCTTTGG-3′ (23mer), reverse primer: 5′-GCGGCCGC-TTATGCACCAGAGTT-3′ (23mer)), mKlf4(forward primer: 5′-GAATTC-ACATGGCTGTCAGCGAC-3′ (23mer), reverse primer: 5′-GCGGCCGC-TTAAAAGTGCCTCTTC-3′ (24mer)). A DNA band was subjected to electrolysis after performing a Polymerase Chain Reaction (PCR), dyed with ethidum bromide, and observed under ultraviolet (UV) light. The observed result can be shown in FIG. 1.

FIG. 1 is a photograph showing a DNA band detected by electrophoresis after performing the PCR. Referring to FIG. 1, it can be seen that bands of four genes were accurately detected.

(4) T-Vector Cloning

In order to carry out cloning in T-vector, only PCR band was eluted using a gel extraction kit (product name: QIAquick gel extraction kit, manufactured by Qiagen), and 1 ul of pGEM-T easy vector, 3 ul of target DNA, 1 ul of ligase buffer, 4 ul of water, and 1 ul of ligase (manufactured by Promega) were mixed together to perform overnight reaction at 16. It could be seen that each of the four genes was cloned using a DNA sequencing device (sequencing, Applied biosystems company's 3730XL Capillary DNA sequencer machine). FIG. 2 is a schematic diagram illustrating a mechanism of pGEM-T easy vector.

2. Sub-Cloning in pENTR4 Vector

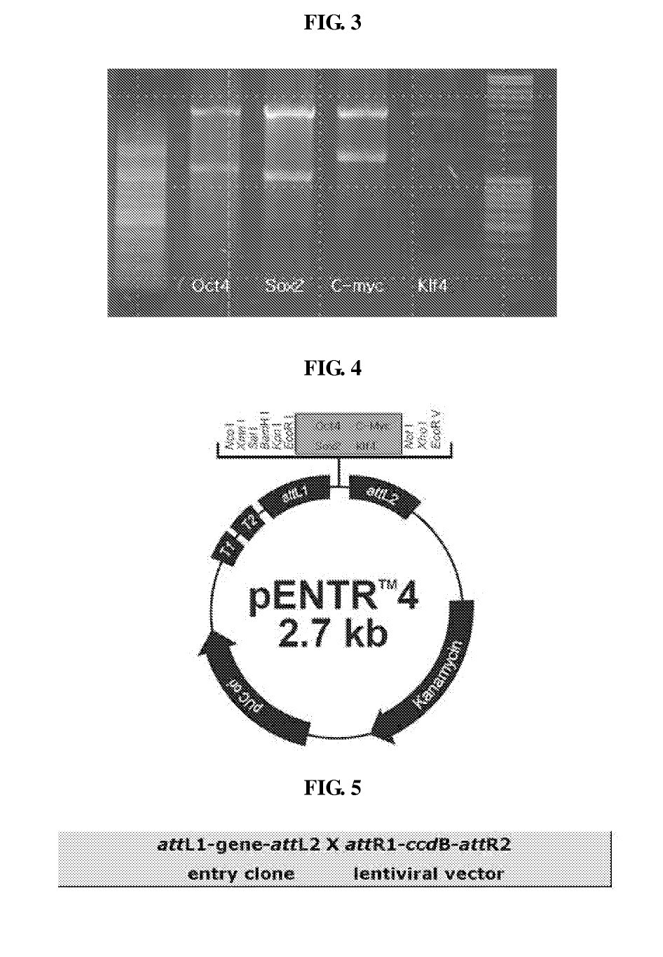

mOct4, mSox2, mC-Myc, and mKlf4 each cloned in T-vector were cut using EcoRI enzyme (see FIG. 3), and then carried out ligation with pENTR4 vector (see FIG. 4). FIG. 3 is an electrophoretic photograph showing genes cloned in T-vector, and FIG. 4 is a schematic diagram illustrating a mechanism of pENTR4 vector.

3. Homologus Recombination with Lentiviral Vector

In order to perform recombination of pENTR4/mOct4, mSox2, mC-myc, and mKlf4 vector and lentiviral vector (see, FIG. 5), each 2 ul of pENTR4/mOct4, mSox2, mC-myc, mKlf4 DNA 4 ul, and lentiviral vector, 2 ul of water, and 2 ul of LR clonase (manufactured by Invitrogen) enzyme were mixed to perform overnight reaction at 20° C.

Then, 1 ul of Proteinase K solution was inserted to be reacted for 10 minutes at 137. Next, the mixture was injected in competent cells, smeared in LB/Apm agar plate, and performed overnight culture at 37. After the overnight culture, a DNA sample was extracted, and observed using the DNA sequencing device (sequencing, Applied biosystems company's 3730XL Capillary DNA sequencer machine), which homologus recombination was carried out. FIG. 5 is a schematic diagram illustrating homologous recombination according to an exemplary embodiment of the present invention. Referring to FIG. 5, a genetic region of an entry cloning vector and a ccdB region of the lentiviral vector were replaced with each other, and thereby the homologous recombination was carried out.

4. Virus Production

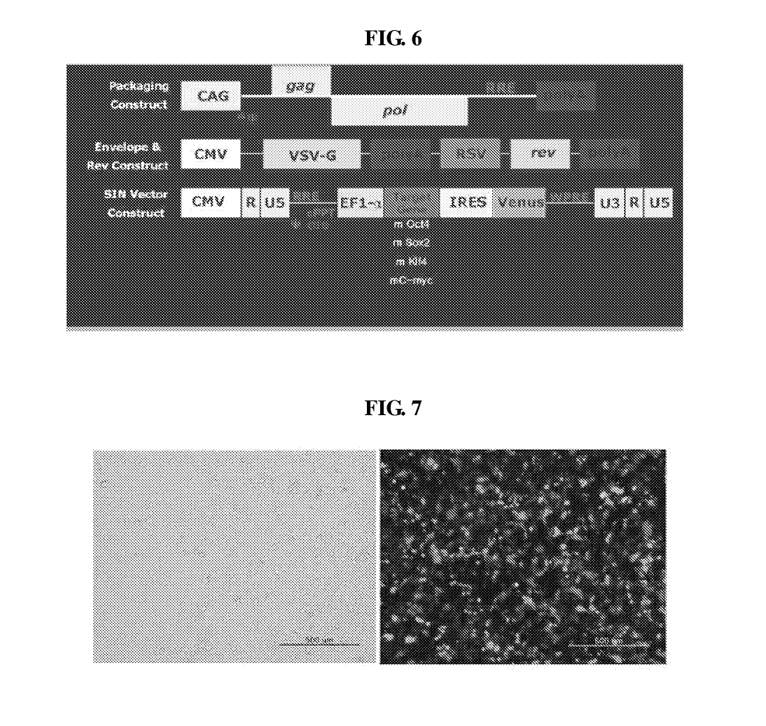

A transient transfection was performed with a 293T cell using calcium phosphate transfection, thereby producing the virus. The calcium phosphate transfection was replaced with a medium (DMEM; manufactured by Sigma) added with a Foetal Bovine Serum (FBS) of 10% 12 to 16 hours after the transfection was performed, and virus particles were produced (See, FIG. 7). Then, 50,000 g of the virus particles were centrifuged for 4 hours at 4° C., thereby concentrating the virus. FIG. 6 is a mimetic diagram illustrating an envelope plasmid, a packaging plasmid, and a target vector each for producing viruses, and FIG. 7 is a microphotograph illustrating a state where a lentiviral vector is infected in a 239T cell.

5. Gene Injection in Mouse Somatic Cells Using Lentiviral Infection

0.5×10⁶ numbered mouse somatic cells prepared in a culture dish of 100 mm the previous day were detached from the culture dish to thereby be floated. The floating of the mouse somatic cells was carried out such that the somatic cells were detached using the triple solution, and solid contents of the somatic cells were separated from the detached somatic cells using centrifugation. 50 ul of each virus concentrated solution corresponding to the respective genes was mixed with a first culture solution at a ratio of 1:1 (200 ul of a virus mixture; 200 ul of the culture solution), reacted with the floated somatic cells for 5 to 10 minutes in a conical tube of 15 ml, and then placed in a culture dish of 100 mm where 5,600 ul of a second culture solution was contained. Gene transfer was performed by cultivating the virus concentrated solution for 24 hours. At the time of injecting the gene, total 6 ml of the culture solution was used, and 0.6 μg/ml of polybrene (manufactured by Sigma) was processed. The somatic cell-culture solution (first culture solution) and the culture solution (second culture solution) used at the time of injecting the gene were obtained by adding each of 0.1 mM of β-mercaptoethanol (manufactured by Sigma), a non-essential amino acid of 1%, 50 U/ml of penicillin, 50 ug/ml of streptomycinm, and FBS of 10% (manufactured by Hyclone) to a DMEM culture solution (No. 11995, manufactured by Invitrogen) where 4.5 g/L of high-glucose, 0.11 g/L of Na-pyruvate, and 2 mM of L-glutamine were contained.

Each of the cells obtained by methods of performing the gene transfer was detached from the culture dish using the triple solution, and placed on five culture dishes of each being 60 mm where STO feeder cells (mouse fibroblast cells) prepared the previous day were contained, to thereby be cultivated. In this instance, a somatic cell-culture solution was used for the used culture solution. The somatic cell-culture solution was obtained by enabling FBS of 15% and 1,000 U/ml of Leukemia inhibitory factor, that is, an undifferentiated inducer required for stem cell maintenance to be contained in the above-mentioned culture solution composition. The virus infection was observed through fluorescence 24 to 48 hours after the cultivation.

### Comparative Example

According to the present Comparative Example, the remaining processes except for the gene transfer process were performed in the same way as the above-described Example in comparison with the Example.

According to the present Comparative Example, the gene transfer was performed on somatic cells attached on the culture dish. Each 50 ul (total 200 ul) of the virus concentrated solution was directly sprayed on 5,800 ul of the culture solution. In this instance, the virus concentrated solution was obtained such that each of the Oct4, Sox2, C-myc, and Klf4 was contained in 0.5×10⁶ numbered mouse somatic cells prepared in the culture dish of 100 mm the previous day.

### Analysis on Characteristics of Somatic Cells

1. Alkaline Phosphatase (AP) Activity Measurement

In order to examine characteristics of an undifferentiated somatic cell colony shaped in a form, activity of the AP widely used as a marker of undifferentiated cells was measured. The colony was fixed for one minute at formaldehyde (manufactured by Sigma) of 4%, washed using Tris-HCl, reacted for 15 minutes with a dye kit (product name: Fast Red Violet/Naphthol AS-BI, manufactured by Chemicon) to thereby be washed, and then a degree of the reaction was observed using a microscope.

2. Verification of Presence/Absence of Gene Expression

(1) Verification of Presence/Absence of Gene Expression Using SSEA-1

The presence/absence of the gene expression was verified using a Stage-Specific Embryonic Antigen 1 (SSEA-1, manufactured by Santacruz) recognizing undifferentiated embryonic stem cells. A colony presumed to be embryonic stem cells was fixed for 15 minutes with paraform-aldehyde (PFA, manufactured by Sigma) of 4%, washed three times using the PBS, and performed non-specific blocking for 30 minutes using a normal goat serum of 10%. Then, the colony was reacted with a first antibody for 6 minutes at 37° C. with a concentration ratio of 1:20. Next, in order to a degree of the reaction, the colony was washed three times using the PBS, and a second antibody (product name: rhodamine (TRITC)-conjugated goat anti-mouse IgM, 1:100, manufactured by Jackson Lab) on which TRITC is attached was processed. For nuclear staining, 4′-6-diamidino-2-phenylindole (DAPI, 1:200, manufactured by Sigma) was processed, reacted for 30 minutes at 37° C., sufficiently washed using the PBS, and then observed using a fluorescence microscope.

(2) Verification of Presence/Absence of Oct-4 Expression

The presence/absence of Oct-4 (ocatamer-binding transcription factor-4) expression specifically expressed in undifferentiated embryonic stem cells was examined. A colony presumed to be embryonic stem cells was fixed for 15 minutes with the PFA of 4%, washed three times using the PBS, penetrated for 10 minutes using a triton X-100 solution (manufactured by Sigma) of 0.2%, and then performed blocking for 30 minutes using the normal goat serum of 10%. Next, the colony was washed using the PBS, and reacted with an Oct-4 antibody (manufactured by Santa Cruz, 1:50) for 60 minutes at 37° C. In order to a degree of the reaction with respect to the Oct-4, a second antibody (product name: TRITC-conjugated goat anti-rabbit IgG, 1:200, manufactured by Jackson Lab) on which TRITC is attached was processed. For nuclear staining, DAPI (1:1,000) was processed, reacted for 30 minutes at 37° C., sufficiently washed using the PBS, and then observed using the fluorescence microscope.

3. Examination of In Vitro Differentiation of Stem Cells

In order to examine in vitro differentiation potency of embryonic stem cells, a plurality of colonies was made into an Embryoid Body (EB) having triploblastic characteristics for three days, attached on a culture dish on which gelatin is coated, and then performed dye by inducing spontaneous differentiation for one week within a culture solution containing a serum. The differentiated cell was fixed for 15 minutes using PFA of 4%, washed using the PBS, penetrated for 10 minutes using the triton X-100 solution of 0.2%, and then performed blocking for one hour using the normal goat serum of 10%. An anti-βIII tubulin monoclonlal antibody (Tuji, 1:200, manufactured by Chemicon) of a nerve cell factor was used for examining ectoderm potency, an anti-α-smooth muscle actin monoclonal antibody (SMA, 1;25, manufactured by Santacruz) was used for examining mesoderm potency, and an anti-α-fetoprotein polyclonal antibody (AFP, 1;200, manufactured by Sigma) was used for examining endoderm potency. Each of the above-mentioned antibodies performed overnight reaction at 4° C. In order to a degree of the reaction with respect to each of the first antibodies, a second antibody (TRITC conjugated goat anti-rabbit IgG, 1:200, manufactured by Jackson Lab) on which TRITC is attached was processed. For nuclear staining, DAPI (1:1,000) was processed, reacted for one hour at a room temperature, sufficiently washed using the PBS, and then observed using the fluorescence microscope.

### Results Analysis

1. Examination of Presence/Absence of Occurrence of Gene Transfer in Mouse Somatic Cells

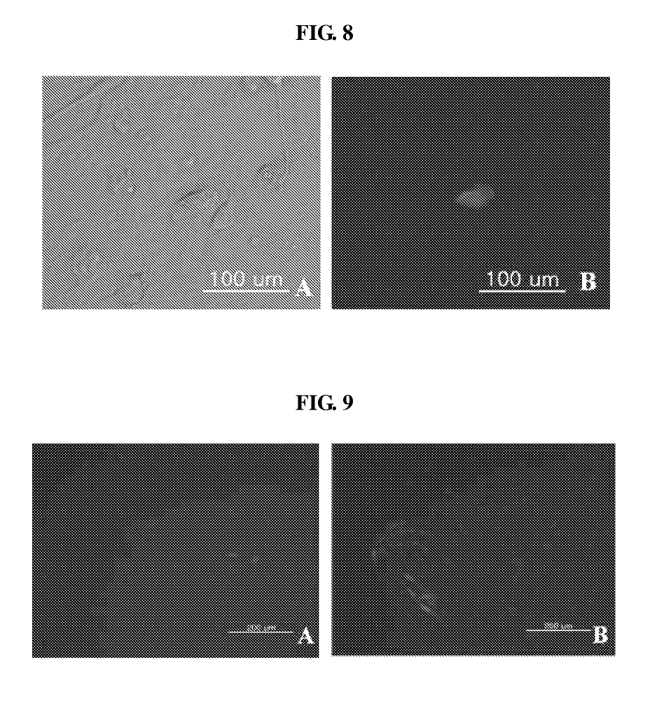

FIG. 8 is a microphotograph (A) and a fluorescence microphotograph (B) each showing stem cells 24 hours after inducing genes. Referring to FIG. 8, it can be seen that a Venus marker-gene was expressed.

FIG. 9 is a fluorescence microphotograph showing stem cells 48 hours after inducing genes by Comparative Example (A) and Example (B). Referring to FIG. 9, it could be found that gene transfer efficiency of the case of somatic cells where the gene transfer was performed by Example was superior to that of the case of somatic cells where the gene transfer was performed by Comparative Example.

2. Production of Induced Pluripotent Stem (iPS) Cells

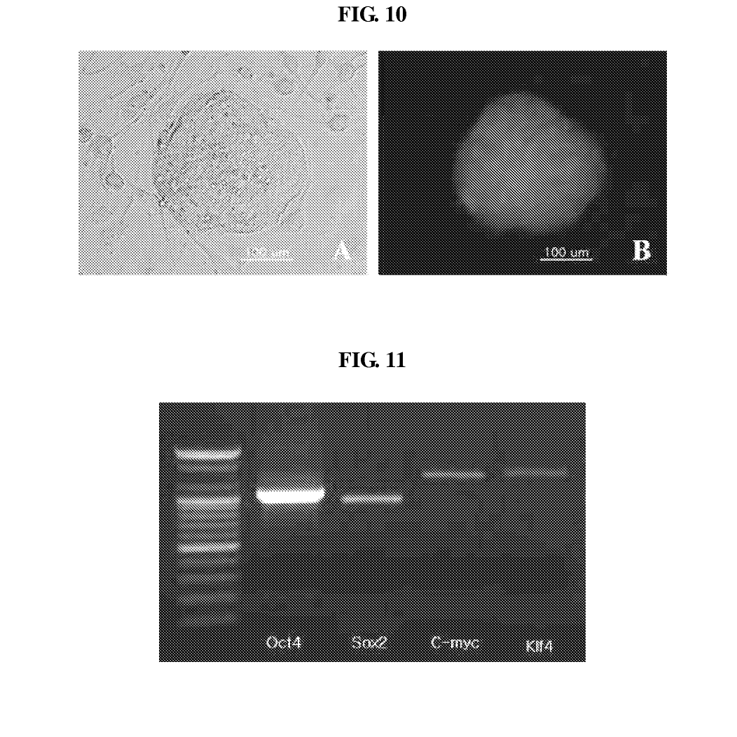

FIG. 10 is a microphotograph (A) and a fluorescence microphotograph (B) each showing stem cells in 10 days after inducing genes. Referring to FIG. 10, it can be seen that stem cells of a colony was established. When comparing a number of colonies formed by the gene induced by Comparative Example (A) and Example (B), respectively, the number of colonies formed by Example (B) was 9.3 times greater than that by Comparative Example (A).

3. Verification of Expression of Four Genes from iPS

FIG. 11 is an electrophoretic photograph showing gene expression within stem cells originated from somatic cells. Referring to FIG. 11, it could be found that four genes, that is, initial four transcription factors were expressed.

4. Examination of Characteristics of iPS

(1) Alkaline Phosphatase (AP) Activity Measurement

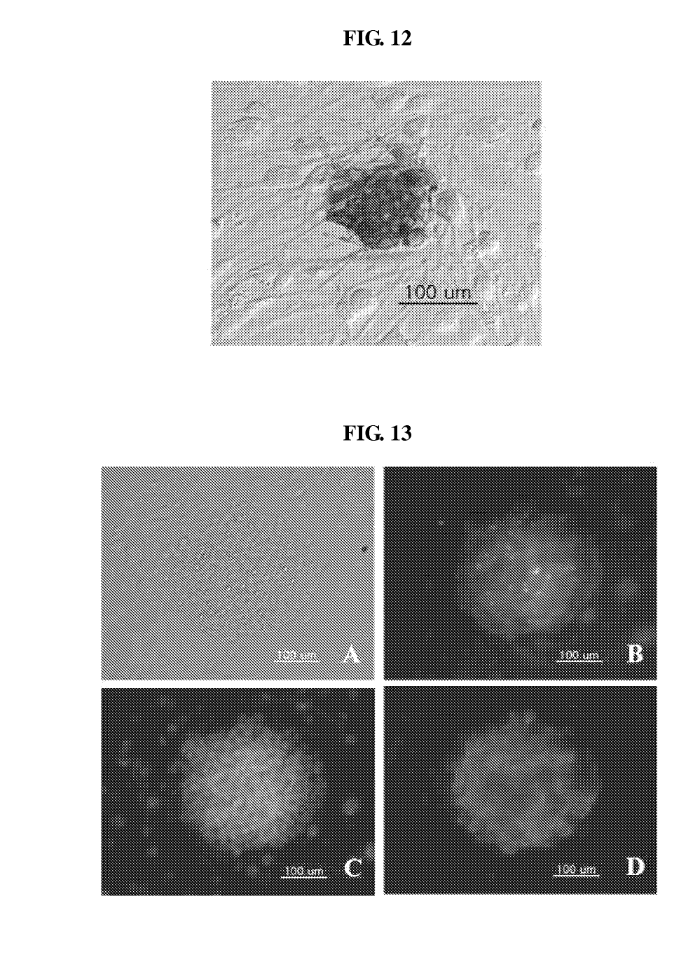

FIG. 12 is a microphotograph showing stem cells in which an Alkaline phosphatase (AP) is activated (substantial microscope-Fast Red Violet/Naphthol AS-BI dye verification).

(2) SSEA-1 Expression

FIG. 13 is microphotographs showing a state of SSEA-1 expression. Referring to FIG. 13, A is a substantial microphotograph, and B is a fluorescence microphotograph where a Venus marker-gene is expressed. Also, C is a fluorescence microphotograph obtained by DAPI dye, and D is a photograph where SSEA-1 is expressed by TRITC dye.

(3) Oct-4 Expression

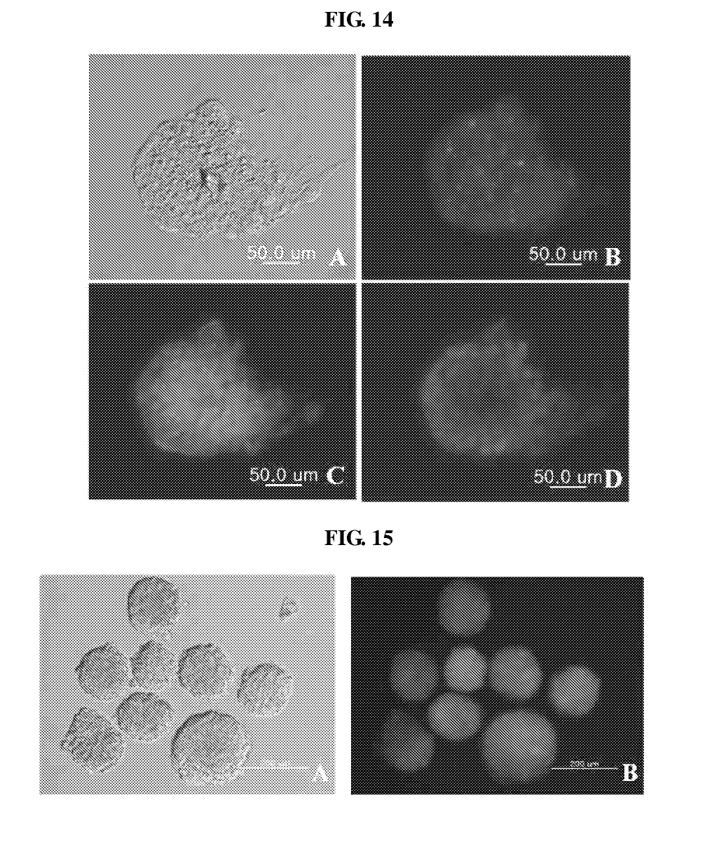

FIG. 14 is microphotographs showing a state of Oct-4 expression. Referring to FIG. 14, A is a substantial microphotograph of iPS, and B is a fluorescence microphotograph where the Venus marker-gene is expressed. Also, C is a fluorescence microphotograph obtained by the DAPI dye, and D is a fluorescence microphotograph where Oct-4 is expressed by the TRITC dye.

(4) Verification of Characteristics of Triploblastic Differentiation of iPS

Induction of Embryoid Body Generation

FIG. 15 is a microphotograph (A) and a fluorescence microphotograph (B) each showing the differentiation of stem cells where differentiation has been induced for three days. Referring to FIG. 15, it could be found that the embryoid body was formed and the Venus marker-gene was expressed.

Induction of Triploblastic Differentiation

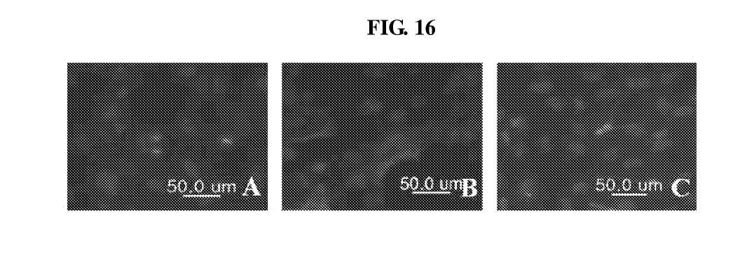

FIG. 16 is fluorescence microphotographs showing the differentiation of stem cells where differentiation has been induced for seven days. Red parts of each of photographs are regions where the TRITC dye is performed, and blue parts thereof are regions where the DAPI dye is performed.

Referring to FIG. 16, it could be found that differentiation of each of endoderm (liver cells, A), mesoderm (muscle cells, B), and ectoderm (nerve cells, C) was performed.

According to the present invention, induced pluripotent stem (iPS) cells may be effectively manufactured without using an egg cell, and thus can be expected to contribute to maximize the process efficiency when the mass production is attained in the future.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

1. A method for manufacturing stem cells, the method comprising:
preparing Oct-4 gene, Sox2 gene, C-myc gene, and Klf-4 gene from mouse embryonic stem cells, and allowing each of the genes to be infected in host cells using a lentiviral vector system to generate viruses in which each of the genes are induced;
concentrating or mixing each of the viruses to prepare a virus concentrated mixture, and mixing the virus concentrated mixture and a first culture solution to prepare a virus solution;
floating mouse somatic cells having been cultivated in advance in a first culture dish, and mixing and reacting the floated somatic cells and the virus solution to prepare a somatic cell-virus mixture;
adding and retaining the somatic cell-virus mixture as is in a second culture dish including a second culture solution to induce the genes in the somatic cells; and
cultivating the somatic cells in which the genes are induced in a third culture dish including a third culture solution.

2. The method of claim 1, wherein the allowing of each of the genes to be infected in host cells includes:
preparing the Oct-4 gene, the Sox2 gene, the C-myc gene, and the Klf-4 gene from the mouse embryonic stem cells to clone the genes in a lentiviral vector, respectively; and
allowing the cloned lentiviral vectors to be infected in the host cells to generate viruses in which the genes are induced by the cloned lentiviral vectors, respectively.

3. The method of claim 1, wherein the concentrating of each of the viruses is achieved by centrifugation.

4. The method of claim 1, wherein the mixing of each of the viruses is performed in such a manner that an amount of each of the viruses is the same.

5. The method of claim 1, wherein the virus concentrated mixture and the first culture solution are mixed with a ratio of about 1:1 to 5.

6. The method of claim 1, wherein the floating of mouse somatic cells includes:
separating the somatic cells from the first culture dish using a cell separation solution; and
centrifuging the separated somatic cells.

7. The method of claim 1, wherein a volume ratio of the somatic cell-virus mixture and the second culture solution is about 1:10 to 20.

8. The method of claim 1, wherein composition of the first and second culture solutions is the same.

9. The method of claim 1, wherein the reacting is performed for about 5 to 15 minutes.
